SEQUENCE LISTING (10 sequences)

SEQ ID NO: 1 - DNA, 2017 bp, Glycine max
tcgttccctctcatgtgttcgacgatttgaaaaaaagaataagaaggagcatgctaaaggaaaggaaatatgatcctaaaatgtattcatgaaattgatacaaaatcaaattgcaacaacattaatttattggaaggaaatgaaatttgtgaaattgaaaaaaaaaaaatcaagtcacatgtgctgaaggaaggagcaacgagatactagtaggatagtggttactaggaaattatatatgaaattttacacctaaggactaaatattaatagaatatgagattatatttttattggaaataaaatatatctagaattaatttttttatcttgtattctcaaaaataattaaatattccgtgaaataaatactcctatctcaatttctttgcctccccatttttttattataaagtttgagacaaataaatgaaattgacataatacacttaaatgaagactatcatttatttttccaaaaccatagaaattgaaggaatggagaggatttttttccatgaaccgtatgtaaagtttttgcataacgaatgggcgaaaatgagttacgtatatgtttgtatttaatttgagttgagtggtgatgtatcgataacataaaataattttatatattcatttaattataaattattatttatataacttttaaataattatcacacatgatacattaatattatgattggaaacgatataaattattttacactggcagtctgttttttatttttttattataaataataagactccaaaaaatagtaataattaagctttatattttccaaattaagttatagttaaatacaaaagaaatcattaataattaggctttatatgctccatcttaaaatacacaataataataaaaaattgcacaaagtaaactaatacagttttgaaatttaaatgtatgactaattaggttgcgtatgctcgataacttaaagctttgattgagttccaaaaaagattatgatttatcttaaagagatcacagttgcagcagtacaatttcatacctgaaaattcagaggaatatttgctattctgattggagtaacattctaaaaattactaacaacattggagaagaaaacataaaaaaattactaacaacatataaagaaattactagcaacatataaaaatattagatatattagatagggtacaagggatactcaacctttctacagaaaacaaaaaaacctgttctgcaccattccactgaccaattaaatctgataaaccttttcctataaataataaacacaccttagtactcctacataattccttcaaacactccatgggatacattgaccattcataaaaagaaaaggaaaaagacttcattctcctattagaccatcatatccacgatctatacttgatcaaatcagaaacacattcaaagttaggactgtcattattgaaaacaattttatttctatgtaaccatagagtgaccattcgcatatggtctctttgtttttgctagggagcaaaattattttctgtaacgtttccttttggcttaagctttattttttttggattttatcaaggtcgactcttgatctttgattccaagcttagttagatataacctctttgtccgttttggattgctattttatgtcttgtatcttcatggattcctcttaaatttctgatgtttcttatatatatacaagggttagagtattccttttacttctgatttgaatcccatgttaataaattttttttgcctgcaaaaaaagtcaacaaagaaagtaggacacttctcacatttaccccttctttcgaaccaaaagcaatgataagtaaatcacaaaacaaataaacaactaacacaaacaacaaccttgcaacttcttggtatataaatacttgctaattcctccttactgatcacaaacttgttttactgcaaacataagttgtcttccaagtttctttgtgtttatatttctgaagttcctaaatc

SEQ ID NO: 2 - DNA, 3578 bp, Glycine max
atggcacacagaacagcaccatcttggtctaccttcacacttaaatggatttatgatgtcttcctcagttttagaggtgaagacactcgtcaaaaattcactggcaatctctacaattctctgtgtgaaaagggagttcatactttcattgatgatgaagggcttagaagaggggaagaaattacaccagctcttctcaatgccattcaaaattcaaggattgccattgttgttttctctaagaactatgcatcctcaactttttgtttggatgaacttgtcaagatccttgagtgcttgaaggaagaaaaaggtcgatcagtttttccaatattttatgatgtggatccatcacatgttcggcatcagaaagggacttattcagaagcattggcaaaacatgaagaaaggttcccagatgacagtgacaaagtgcaaaaatggaggaaggctttatatgaagcagccaatctatcaggctggcatttccaacatgggtattctatacttcctttttattgtatgtgttataaactgatctctttttacatttgcctaataattttcatattaattactcacaaagaaagaaagcatagtaaatcacattgatctatcttgaacggtgaaatttaagaaatggagacaataaatttatgagattaattctgattgaaatgagtaaactaaacacaaatttgaagctttagtttcaattcacatgttttatacaatgggcagggagctagaatataagtccatccgaaaaattgttaaagaggtctataaaaggattagttgcattcctttacacattgctgataatccaattgggctagagcatgcagtgctagaagtgaaatctctccttggacatggttctgatgtcaacattataggaatttatggtattggtggtatcggtaaaaccacaatttctcgtgccgtgtataacttgatttgtagtcaatttgaaggcacatgttttctccctgacattagagaaaaggcaatcaataagcaaggtcttgtccaactccaagaaatgttactttctgaagtactcaagaagaaacatatcaaagtgggtgatgttaacagaggaattccaataataaaaaggaggcttgaaaaaaagaaggttcttctggttttagatgatgttgacaaattagagcagttaaaggttcttgcaggagaaagtcgttggtttggcaatggaagtataatcattatcaccacaagagacaaacacttgctagcgactcatggggtggtaaaaatatatgatgttaaaccattaaatgttgcaaaggctcttgaattgtttaactggtgtgcctttaaaaatcataaagctgatccactttatgtgaatattgctaaccgtgcagtttcttatgcgtgtggcattccattggctctggaagtgataggttctcatttatttggaaaaagtttgaatgaatgtaattctgcattggataaatatgaaagaattcctcatgaaaagatccatgaaatacttaaagtaagctatgatggtttggaagaaaatgagaagcaaattttccttgacattgcttgtttcttcaatacttgtggtgtgggctacgtcacatcagtgctacgtgctcatggttttcatgtaaaagatggtttgagagtgttggttgacagatctctcttaaagattgatgcttctggttgtgtgagaatgcatgatctaattcgagacacaggcagagaaattgtaaggcaagaatcaacagttgagcctggcagacgtagtaggttatggtttgaagaggacattgttcatgttttggaagaaaatacggtatgttagaaattacgaacatctctatgctatttctttgcttttcattcctttattaaaataacaattttactgaatacatgataaattttgggattctttcccctgttatctatttaatttttatatgatgatgagattgctaatgtgcacatttttgtggctaaattgtgctttgggcatggaagggaactgataaaatagaattcataaagcttgaagggtacaacaacatacaagtgcaatggaatggaaaagccttgaaggagatgaagaacctgaggattttgattattgaaaatacaaccttttctacaggccccgagcatctgccaaatagtttgagagtgctagattggagttgctacccttcaccatctttgcctgctgatttcaatcctaaacgagttgagctactccttatgcctgaaagttgccttcaaatcttccaaccatacaatgtatgtatcagtttcgaagttattatttttaacatgaatttctactttaaaaaaaaaaaaaaaaacacgcacctcacacacaacttgagtcattgtcttttaatttgcagatgtttgaatcattgtctgtgttaagcattgaagattgccagttcttaacagatctaccaagcctaagagaagtaccacttttagcatatttgtgtattgataattgcaccaatttggttaaaattgatggttcaattggctttcttgataagctccaactcttgagtgctaaacggtgtagcaagctaaaaattctagcaccctgcgtcatgttgccatctcttgaaattttggaccttcgggggtgcacatgtcttgatagcttcccggaagtattgggaaagatggagaacataaaagaaatatatttagacgagacagccatagagacactgccatgttcaattggaaattttgttgggcttcaacttttgtccctgaggaaatgcggaaggcttcatcagctaccaggtagtatatgtatattgccgaaagttaaagtgattttcggttttgggcatgtggtatatcgattttgggaagagaaccaatatgaacaagagttgagcttagaagtgtctccaaggtccatggttgttgttgatggtgacctggattttacatatattgacatgtattatccacacataagtccaaataatgtcatccaagtatgcagtcctaatctgctcttgcatcatgatttcattttgttgttcaaaaagcttgcaagcaaggatgggagcagaggcaggaaatcgtcaatgcatttctcgtttcgaaacaaattccccaagattgcggtatgttgttctgtattgcctagtttgaaaattaccatgatattgattttgaagttgggcgtactcatcaacggcactaagatatttagttcatcatgtaactacttatttacacaaacatggaacccaaaactttggtgtgatctagaaagcaaagcagaggggatgttttcagagcaagaatggaatgaagctgagattttgtttgagttgggataccccacaccatgtgctggattaacaatgtcgctccacaactttgctgagggacgtctaaattggaccctatttggtgtctacgagaagggaaataataaggaggatattaaatttaaagatcccatgtcaaccttttcattctgtaacatgctcatggaacctccctcttcattacctagttgtttgtactatgttgttaaatga

SEQ ID NO: 3 - protein, 1011 aa, Glycine max
MAHRTAPSWSTFTLKWIYDVFLSFRGEDTRQKFTGNLYNSLCEKGVHTFIDDEGLRRGEEITPALLNAIQNSRIAIVVFSKNYASSTFCLDELVKILECLKEEKGRSVFPIFYDVDPSHVRHQKGTYSEALAKHEERFPDDSDKVQKWRKALYEAANLSGWHFQHGELEYKSIRKIVKEVYKRISCIPLHIADNPIGLEHAVLEVKSLLGHGSDVNIIGIYGIGGIGKTTISRAVYNLICSQFEGTCFLPDIREKAINKQGLVQLQEMLLSEVLKKKHIKVGDVNRGIPIIKRRLEKKKVLLVLDDVDKLEQLKVLAGESRWFGNGSIIIITTRDKHLLATHGVVKIYDVKPLNVAKALELFNWCAFKNHKADPLYVNIANRAVSYACGIPLALEVIGSHLFGKSLNECNSALDKYERIPHEKIHEILKVSYDGLEENEKQIFLDIACFFNTCGVGYVTSVLRAHGFHVKDGLRVLVDRSLLKIDASGCVRMHDLIRDTGREIVRQESTVEPGRRSRLWFEEDIVHVLEENTGTDKIEFIKLEGYNNIQVQWNGKALKEMKNLRILIIENTTFSTGPEHLPNSLRVLDWSCYPSPSLPADFNPKRVELLLMPESCLQIFQPYNMFESLSVLSIEDCQFLTDLPSLREVPLLAYLCIDNCTNLVKIDGSIGFLDKLQLLSAKRCSKLKILAPCVMLPSLEILDLRGCTCLDSFPEVLGKMENIKEIYLDETAIETLPCSIGNFVGLQLLSLRKCGRLHQLPGSICILPKVKVIFGFGHVVYRFWEENQYEQELSLEVSPRSMVVVDGDLDFTYIDMYYPHISPNNVIQVCSPNLLLHHDFILLFKKLASKDGSRGRKSSMHFSFRNKFPKIAVCCSVLPSLKITMILILKLGVLINGTKIFSSSCNYLFTQTWNPKLWCDLESKAEGMFSEQEWNEAEILFELGYPTPCAGLTMSLHNFAEGRLNWTLFGVYEKGNNKEDIKFKDPMSTFSFCNMLMEPPSSLPSCLYYVVK

SEQ ID NO: 4 - DNA, 3755 bp, Glycine max
atggcacacagaacagcaccatcttggtctaccttcacacttaaatggatttatgatgtcttcctcagttttagaggtgaagacactcgtcaaaaattcactggcaatctctacaattctctgtgtgaaaagggagttcatactttcattgatgatgaagggcttagaagaggggaagaaattacaccagctcttctcaatgccattcaaaattcaaggattgccattgttgttttctctaagaactatgcatcctcaactttttgtttggataaacttgtcaagatccttgagtgcttgaaggaagaaaaaggtcgatcagtttttccaatattttatgatgtggatccatcacatgttcggcatcagaaagggacttattcagaagcattggcaaaacatgaagaaaggttcccagatgacagtgacaaagtgcaaaaatggaggaaggctttatatgaagcagccaatctatcaggctggcatttccaacatgggtattctatacttcctttttattgtatgtgttataaactgatctctttttacatttgcctaataattttcatattaattactcacaaagaaagaaagcatagtaaatcacattgatctatcttgaacggtgaaatttaagaaatggagacaataaatttatgagattaattctgattgaaatgagtaaactaaacacaaatttgaagctttagtttcaattcacatgttttatacaatgggcagggagctagaatataagtccatccgaaaaattgttaaagaggtctataaaaggattagttgcattcctttacacattgctgataatccaattgggctagagcatgcagtgctagaagtgaaatctctccttggacatggttctgatgtcaacattataggaatttatggtattggtggtatcggtaaaaccacaatttctcgtgccgtgtataacttgatttgtagtcaatttgaaggcacatgttttctccttgacattagagaaaaggcaatcaataagcaaggtcttgtccaactccaagaaatgttactttctgaagtactcaagaagaaacatatcaaagtgggtgatgttaacagaggaattccaataataaaaaggaggcttgaaaaaaagaaggttcttctggttttagatgatgttgacaaattagagcagttaaaggttcttgcaggagaaagtcgttggtttggcaatggaagtataatcattatcaccacaagagacaaacacttgctagcgactcatggggtggtaaaaatatatgatgttaaaccattaaatgttgcaaaggctcttgaattgtttaactggtgtgcctttaaaaatcataaagctgatccactttatgtgaatattgctaaccgtgcagtttcttatgcgtgtggcattccattggctctggaagtgataggttctcatttatttggaaaaagtttgaatgaatgtaattctgcattggagggcgagccctggtgcagcggtaaagttgtgccttggtgacttgttggtcatgggttcgaatccggaaacagcctctttgcatatgcaagggtaagactgcgtacaatatccctccctcataccttcgcatagcgaagagcctctgggcaatggggtacgaatgtaattctgcattggataaatatgaaagaattcctcatgaaaagatccatgaaatacttaaagtaagctatgatggtttggaagaaaatgagaagcaaattttccttgacattgcttgtttcttcaatacttgtggtgtgggctacgtcacatcagtgctacgtgctcatggttttcatgtaaaagatggtttgagagtgttggttgacagatctctcttaaagattgatgcttctggttgtgtgagaatgcatgatctaattcgagacacaggcagagaaattgtaaggcaagaatcaacagttgagcctggcagacgtagtaggttatggtttgaagaggacattgttcatgttttggaagaaaatacggtatgttagaaattacgaacatctctatgctatttctttgcttttcattcctttattaaaataacaattttactgaatacatgataatttttgggattctttcccctgttatctatttaatttttatatgatgatgagattgctaatgtgcacatttttgtggctaaattgtgctttgggcatggaagggaactgataaaatagaattcataaagcttgaagggtacaacaacatacaagtgcaatggaatggaaaagccttgaaggagatgaagaacctgaggattttgattattgaaaatacaaccttttctacaggccccgagcatctgccaaatagtttgagagtgctagattggagttgctacccttcaccatctttgcctgctgatttcaatcctaaacgagttgagctactccttatgcctgaaagttgccttcaaatcttccaaccatacaatgtatgtatcagtttcgaagttattatttttaacatgaatttctactttaaaaaaaaaaaaaaacacgcacctcacacacaacttgagtcattgtcttttaatttgcagatgtttgaatcattgtctgtgttaagcattgaagattgccagttcttaacagatctaccaagcctacgagaagtaccacttttagcatatttgtgtattgataattgcaccaatttggttaaaattgatggttcaattggctttcttgataagctccaactcttgagtgctaaacggtgtagcaagctaaaaattctagcaccctgcgtcatgttgccatctcttgaaattttggaccttcgggggtgcacatgtcttgatagcttcccggaagtattgggaaagatggagaacataaaagaaatatatttagacgagacagccatagagacactgccatgttcaattggaaattttgttgggcttcaacttttgtccctgaggaaatgcggaaggcttcatcagctaccaggtagtatatgtatattgccgaaagttaaagtgattttcggttttgggcatgtggtatatcgattttgggaagagaaccaatatgaacaagagttgagcttagaagtgtctccaaggtccatggttgttgttgatggtgacctggattttacatatattgacatgtattatccacacataagtccaaataatgtcatccaagtatgcagtcctaatctgctcttgcatcatgatttcattttgttgttcaaaaagcttgcaagcaaggatgggagcagatgcaggaaatcgtcaatgcatttctcgtttcgaaacaaattccccaagattgcggtatgttgttctgtattgcctagtttgaaaattaccatgatattgattttgaagttgggcgtactcatcaacggcactaagatatttagttcatcatgtaactacttatttacacaaacatggaacccaaaactttggtgtgatctagaaagcaaagcagaggggatgttttcagagcaagaatggaatgaagctgagattttgtttgagttgggataccccacaccatgtgctggattaacaatgtcgctccacaactttgctgagggacgtctaaattggaccctatttggtgtctacgaggagggaaataataaggaggatattaaatttaaagatcccatgtcaaccttttcattctgtaacatgctcatggaacctccctcttcattacctagttgtttgtactatgttgttaaatga

SEQ ID NO: 5 - DNA, 3755 bp, Glycine max
atggcacacagaacagcaccatcttggtctaccttcacaattaaatggatttatgatgtcttcctcagttttagaggtgaagacactcgtcaaaaattcactggcaatctctacaattctctgtgtgaaaagggagttcatactttcattgatgatgaagggcttagaagaggggaagaaattacaccagctcttctcaatgccattcaaaattcaaggattgccattgttgttttctctaagaactatgcatcctcaactttttgtttggatgaacttgtcaagatccttgagtgcttgaaggaagaaaaaggtcgatcagtttttccaatattttatgatgtggatccatcacatgttcggcatcagaaagggacttattcagaagcattggcaaaacatgaagaaaggttcccagatgacagtgacaaagtgcaaaaatggaggaaggctttatatgaagcagccaatctatcaggctggcatttccaacatgggtattctatacttcctttttattgtatgtgttataaactgatctctttttacatttgcctaataattttcatattaattactcacaaagaaagaaagcatagtaaatcacattgatctatcttgaacggtgaaatttaagaaatggagacaataaatttatgagattaattctgattgaaatgagtaaactaaacacaaatttgaagctttagtttcaattcacatgttttatacaatgggcagggagctagaatataagtccatccgaaaaattgttaaagaggtctataaaaggattagttgcattcctttacacattgctgataatccaattgggctagagcatgcagtgctagaagtgaaatctctccttggacatggttctgatgtcaacattataggaatttatggtattggtggtatcggtaaaaccacaatttctcgtgccgtgtataacttgatttgtagtcaatttgaaggcacatgttttctccctgacattagagaaaaggcaatcaataagcaaggtcttgtccaactccaagaaatgttactttctgaagtactcaagaagaaacatatcaaagtgggtgatgttaacagaggaattccaataataaaaaggaggcttgaaaaaaagaaggttcttctggttttagatgatgttgacaaattagagcagttaaaggttcttgcaggagaaagtcgttggtttggcaatggaagtataatcattatcaccacaagagacaaacacttgctagcgactcatggggtggtaaaaatatatgatgttaaaccattaaatgttgcaaaggctcttgaattgtttaactggtgtgcctttaaaaatcataaagctgatccactttatgtgaatattgctaaccgtgcagtttcttatgcgtgtggcattccattggctctggaagtgataggttctcatttatttggaaaaagtttgaatgaatgtaattctgcattggagggcgagccctggtgcagcggtaaagttgtgtcttggtgacttgttggtcatgggttcgaatccggaaacagcctctttgcatatgcaagggtaaggctgcgtacaatatccctcccccataccttcgcatagcgaagagcctctgggcaatggggtacgaatgtaattctgcattggataaatatgaaagaattcctcatgaaaagatccatgaaatacttaaagtaagctatgatggtttggaagaaaatgagaagcaaattttccttgacattgcttgtttcttcaatacttgtggtgtgggctacgtcacatcagtgctacgtgctcatggttttcatgtaaaagatggtttgagagtgttggttgacagatctctcttaaagattgatgcttctggttgtgtgagaatgcatgatctaattcgagacacaggcagagaaattgtaaggcaagaatcaacagttgagcctggcagacgtagtaggttatggtttgaagaggacattgttcatgttttggaagaaaatacggtatgttagaaattacgaacatctctatgctatttctttgcttttcattcctttattaaaataacaattttactgaatacatgataaattttgggattctttcccctgttatctatttaatttttatatgatgatgagattgctaatgtgcacatttttgtggctaaattgtgctttgggcatggaagggaactgataaaatagaattcataaagcttgaagggtacaacaacatacaagtgcaatggaatggaaaagccttgaaggagatgaagaacctgaggattttgattattgaaaatacaaccttttctacaggccccgagcatctgccaaatagtttgagagtgctagattggagttgctacccttcaccatctttgcctgctgatttcaatcctaaacgagttgagctactccttatgcctgaaagttgccttcaaatcttccaaccatacaatgtatgtatcagtttcgaagttattatttttaacatgaatttctacttaaaaaaaaaaaaaaaacacgcacctcacacacaacttgagtcattgtcttttaatttgcagatgtttgaatcattgtctgtgttaagcattgaagattgccagttcttaacagatctaccaagcctacgagaagtaccacttttagcatatttgtgtattgataattgcaccaatttggttaaaattgatggttcaattggctttcttgataagctccaactcttgagtgctaaacggtgtagcaagctaaaaattctagcaccctgcgtcatgttgccatctcttgaaattttggaccttcgggggtgcacatgtcttgatagcttcccggaagtattgggaaagatggagaacataaaagaaatatatttagacgagacagccatagagacactgccatgttcaattggaaattttgttgggcttcaacttttgtccctgaggaaatgcggaaggcttcatcagctaccaggtagtatatgtatattgccgaaagttaaagtgattttcggttttgggcatgtggtatatcgattttgggaagagaaccaatatgaacaagagttgagcttagaagtgtctccaaggtccatggttgttgttgatggtgacctggattttacatatattgacatgtattatccacacataagtccaaataatgtcatccaagtatgcagtcctaatctgctcttgcatcatgatttcattttgttgttcaaaaagcttgcaagcaaggatgggagcagatgcaggaaatcgtcaatgcatttctcgtttcgaaacaaattccccaagattgcggtatgttgttctgtattgcctagtttgaaaattaccatgatattgattttgaagttgggcgtactcatcaacggcactaagatatttagttcatcatgtaactacttatttacacaaacatggaacccaaaactttggtgtgatctagaaagcaaagcagaggggatgttttcagagcaagaatggaatgaagctgagattttgtttgagttgggataccccacaccatgtgctggattaacaatgtcgctccacaactttgctgagggacgtctaaattggaccctattcggtgtctacgaggagggaaataataaggaggatattaaatttaaagatcccatgtcaaccttttcattctgtaacatgctcatggaacctccctcttcattacctagttgtttgtactatgttgttaaatga

SEQ ID NO: 6 - DNA, 3756 bp, Glycine max
atggcacacagaacagcaccatcttggtctaccttcacacttaaatggatttatgatgtcttcctcagttttagaggtgaagacactcgtcaaaaattcactggcaatctctacaattctctgtgtgaaaagggagttcatactttcattgatgatgaagggcttagaagaggggaagaaattacaccagctcttctcaatgccattcaaaattcaaggattgccattgttgttttctctaagaactatgcatcctcaactttttgtttggatgaacttgtcaagatccttgagtgcttgaaggaagaaaaaggtcgatcagtttttccaatattttatgatgtggatccatcacatgttcggcatcagaaagggacttattcagaagcattggcaaaacatgaagaaaggttcccagatgacagtgacaaagtgcaaaaatggaggaaggctttatatgaagcagccaatctatcaggctggcatttccaacatgggtattctatacttcctttttattgtatgtgttataaactgatctctttttacatttgcctaataattttcatattaattactcacaaagaaagaaagcatagtaaatcacattgatctatcttgaacggtgaaatttaagaaatggagacaataaatttatgagattaattctgattgaaatgagtaaactaaacacaaatttgaagctttagtttcaattcacatgttttatacaatgggcagggagctagaatataagtccatccgaaaatttgttaaagaggtctataaaaggattagttgcattcctttacacattgctgataatccaattgggctagagcatgcagtgctagaagtgaaatctctccttggacatggttctgatgtcaacattataggaatttatggtattggtggtatcggtaaaaccacaatttctcgtgccgtgtataacttgatttgtagtcaatttgaaggcacatgttttctccctgacattagagaaaaggcaatcaataagcaaggtcttgtccaactccaagaaatgttactttctgaagtactcaagaagaaacatatcaaagtgggtgatgttaacagaggaattccaataataaaaaggaggcttgaaaaaaagaaggttcttctggttttagatgatgttgacaaattagagcagttaaaggttcttgcaggagaaagtcgttggtttggcaatggaagtataatcattatcaccacaagagacaaacacttgctagcgactcatggggtggtaaaaatatatgatgttaaaccattaaatgttgcaaaggctcttgaattgtttaactggtgtgcctttaaaaatcataaagctgatccactttatgtgaatattgctaaccgtgcagtttcttatgcgtgtggcattccattggctctggaagtgataggttctcatttatttggaaaaagtttgaatgaatgtaattctgtattggagggcgagccctggtgcagcggtaaagttgtgccttggtgacttgttggtcatgggttcgaatccggaaacagcctctttgcatatgcaagggtaaggctgcgtacaatatccctccctcataccttcgcatagcgaagagcctctaggcaatggggtacgaatgtaattctgcattggataaatatgaaagaattcctcatgaaaagatccatgaaatacttaaagtaagctatgatggtttggaagaaaatgagaagcaaattttccttgacattgcttgtttcttcaatacttgtggtgtgggctacgtcacatcagtgctacgtgctcatggttttcatgtaaaagatggtttgagagtgttggttgacagatctctcttaaagattgatgcttctggttgtgtgagaatgcatgatctaattcgagacacaggcagagaaattgtaaggcaagaatcaacagttgagcctggcagacgtagtaggttatggtttgaagaggacattgttcatgttttggaagaaaatacggtatgttagaaattacgaacatctctatgctatttctttgcttttcattcctttattaaaataacaattttactgaatacatgataaattttgggattctttcccctgttatctatttaatttttatatgatgatgagattgctaatgtgcacatttttgtggctaaattgtgctttgggcatggaagggaactgataaaatagaattcataaagcttgaagggtacaacaacatacaagtgcaatggaatggaaaaaccttgaaggagatgaagaacctgaggattttgattattgaaaatacaaccttttctacaggccccgagcatctgccaaatagtttgagagtgctagattggagttgctacccttcaccatctttgcctgctgatttcaatcctaaacgagttgagctactccttatgcctgaaagttgccttcaaatcttccaaccatacaatgtatgtatcagtttcgaagttattatttttaacatgaatttctactttaaaaaaaaaaaaaaaacacgcacctcacacacaacttgagtcattgtcttttaatttgcagatgtttgaatcattgtctgtgttaagcattgaagattgccagttcttaacagatctaccaagcctaagagaagtaccacttttagcatatttgtgtattgataattgcaccaatttggttaaaattgaaggttcaattggctttcttgataagctccaactcttgagtgctaaacggtgtagcaagctaaaaattctagcaccctgcgtcatgttgccatctcttgaaattttggaccttcgggggtgcacatgtcttgatagcttcccggaagtattgggaaagatggagaacataaaagaaatatatttagacgagacagccatagagacactgccatgttcaattggaaattttgttgggcttcaacttttgtccctgaggaaatgcggaaggcttcatcagctaccaggtagtatatgtatattgccgaaagttaaagtgattttcggttttgggcatgtggtatatcgattttgggaagagaaccaatatgaacaagagttgagcttagaagtgtctccaaggtccatggttgttgttgatggtgacctggattttacatatattgacatgtattatccacacataagtccaaataatgtcatccaagtatgcagtcctaatctgctcttgcatcatgatttcattttgttgttcaaaaagcttgcaagcaaggatgggagcagaggcaggaaatcgtcaatgcatttctcgtttcgaaacaaattccccaagattgcggtatgttgttctgtattgcctagtttgaaaattaccatgatattgattttgaagttgggcgtactcatcaacggcactaagatatttagttcatcatgtagctacttatttacacaaacatggaacccaaaactttggtgtgatctagaaagcaaagcagaggggatgttttcagagcaagaatggaatgaagctgagattttgtttgagttgggataccccacaccatgtgctggattaacaatgtcgctccacaactttgctgagggacgtctaaattggaccctatttggtgtctacgaggagggaaataataaggaggatattaaatttaaagatcccatgtcaaccttttcattctgtaacatgctcatggaacctccctcttcattacctagttgtttgtactatgttgttaaatga

SEQ ID NO: 7 - DNA, 3756 bp, Glycine max
atggcacacagaacagcaccatcttggtctaccttcacacttaaatggatttatgatgtcttcctcagttttagaggtgaagacactcgtcaaaaattcactggcaatctctacaattctctgtgtgaaaagggagttcatactttcattgatgatgaagggcttagaagaggggaagaaattacaccagctcttctcaatgccattcaaaattcaaggattgccattgttgttttctctaagaactatgcatcctcaactttttgtttggatgaacttgtcaagatccttgagtgcttgaaggaagaaaaaggtcgatcagtttttccaatattttatgatgtggatccatcacatgttcggcatcagaaagggacttattcagaagcattggcaaaacatgaagaaaggttcccagatgacagtgacaaagtgcaaaaatggaggaaggctttatatgaagcagccaatctatcaggctggcatttccaacatgggtattctatacttcctttttattgtatgtgttataaactgatctctttttacatttgcctaataattttcatattaattactcacaaagaaagaaagcatagtaaatcacattgatctatcttgaacggtgaaatttaagaaatggagacaataaatttatgagattaattctgattgaaatgagtaaactaaacacaaatttgaagctttagtttcaattcacatgttttatacaatgggcagggagctagaatataagtccatccgaaaaattgttaaagaggtctataaaaggattagttgcattcctttacacattgctgataatccaattgggctagagcatgcagtgctagaagtgaaatctctccttggacatggttctgatgtcaacattataggaatttatggtattggtggtatcggtaaaaccacaatttctcgtgccgtgtataacttgatttgtagtcaatttgaaggcacatgttttctccctgacattagagaaaaggcaatcaataagcaaggtcttgtccaactccaagaaatgttactttctgaagtactcaagaagaaacatatcaaagtgggtgatgttaacagaggaattccaataataaaaaggaggcttgaaaaaaagaaggttcttctggttttagatgatgttgacaaattagagcagttaaaggttcttgcaggagaaagtcgttggtttggcaatggaagtataatcattatcaccacaagagacaaacacttgctagcgactcatggggtggtaaaaatatatgatgttaaaccattaaatgttgcaaaggctcttgaattgtttaactggtgtgcctttaaaaatcataaagctgatccactttatgtgaatattgctaaccgtgcagtttcttatgcgtgtggcattccattggctctggaagtgataggttctcatttatttggaaaaagtttgaatgaatgtaattatgtattggagggcgagccctggtgcagcggtaaagttgtgccttggtgacttgttggtcatgggttcgaatccggaaacagcctctttgcatatgcaagggtaaggctgcgtacaatatccctcccccataccttcgcatagcgaagagcctctaggcaatggggtgcgaatgtaattctgcattggataaatatgaaagaattcctcatgaaaagatccatgaaatacttaaagtaagctatgatggtttggaagaaaatgagaagcaaattttccttgacattgcttgtttcttcaatacttgtggtgtgggctacgtcacatcagtgctacgtgctcatggttttcatgtaaaagatggtttgagagtgttggttgacagatctctcttaaagattgatgcttctggttgtgtgagaatgcatgatctaattcgagacacaggcagagaaattgtaaggcaagaatcaacagttgagcctggcagacgtagtaggttatggtttgaagaggacattgttcatgttttggaagaaaatacggtatgttagaaattacgaacatctctatgctatttctttgcttttcattcctttattaaaataacaattttactgaatacatgataaattttgggattctttcccctgttatctatttaatttttatatgatgatgagattgctaatgtgcacatttttgtggctaaattgtgctttgggcatggaagggaactgataaaatagaattcataaagcttgaagggtacaacaacatacaagtgcaatggaatggaaaaaccttgaaggagatgaagaacctgaggattttgattattgaaaatacaaccttttctacaggccccgagcatctgccaaatagtttgagagtgctagattggagttgctacccttcaccatctttgcctgctgatttcaatcctaaacgagttgagctactccttatgcctgaaagttgccttcaaatcttccaaccatacaatgtatgtatcagtttcgaagttattatttttaacatgaatttctactttaaaaaaaaaaaaaaaacacgcacctcacacacaacttgagtcattgtcttttaatttgcagatgtttgaatcattgtctgtgttaagcattgaagattgccagttcttaacagatctaccaagcctaagagaagtaccacttttagcatatttgtgtattgataattgcaccaatttggttaaaattgaaggttcaattggctttcttgataagctccaactcttgagtgctaaacggtgtagcaagctaaaaattctagcaccctgcgtcatgttgccatctcttgaaattttggaccttcgggggtgcacatgtcttgatagcttcccggaagtattgggaaagatggagaacataaaagaaatatatttagacgagacagccatagagacactgccatgttcaattggaaattttgttgggcttcaacttttgtccctgaggaaatgcggaaggcttcatcagctaccaggtagtatatgtatattgccgaaagttaaagtgattttcggttttgggcatgtggtatatcgattttgggaagagaaccaatatgaacaagagttgagcttagaagtgtctccaaggtccatggttgttgttgatggtgacctggattttacatatattgacatgtattatccacacataagtccaaataatgtcatccaagtatgcagtcctaatctgctcttgcatcatgatttcattttgttgttcaaaaagcttgcaagcaaggatgggagcagatgcaggaaatcgtcaatgcatttctcgtttcgaaacaaattccccaagattgcggtatgttgttctgtattgcctagttttaaaattaccatgatattgattttgaagttgggcgtactcatcaacggcactaagatatttagttcatcatgtagctacttatttacacaaacatggaacccaaaactttggtgtgatctagaaagcaaagcagaggggatgttttcagagcaagaatggaatgaagctgagattttgtttgagttgcgataccccacaccatgtgctggattaacaatgtcgctccacaactttgctgagggacgtctaaattggaccctatttggtgtctacgaggagggaaataataaggaggatattaaatttaaagatcccatgtcaaccttttcattctgtaacatgctcatggaacctccctcttcattacctagttgtttgtactatgttgttaaatga

SEQ ID NO: 8 - DNA, 3754 bp, Glycine max
atggcacacagaacagcaccatcttggtctaccttcacacttaaatggatttatgatgtcttcctcagttttagaggtgaagacactcgtcaaaaattcactggcaatctctacaattctctgtgtgaaaagggagttcatactttcattgatgatgaagggcttagaagaggggaagaaattacaccagctcttctcaatgccattcaaaattcaaggattgccattgttgttttctctaagaactatgcatcctcaactttttgtttggataaacttgtcaagatccttgagtgcttgaaggaagaaaaaggtcgatcagtttttccaatattttatgatgtggatccatcacatgttcggcatcagaaagggacttattcagaagcattggcaaaacatgaagaaaggttcccagatgacagtgacaaagtgcaaaaatggaggaaggctttatatgaagcagccaatctatcaggctggcatttccaacatgggtattctatacttcctttttattgtatgtgttataaactgatctctttttacatttgcctaataattttcatattaattactcacaaagaaagaaagcatagtaaatcacattgatctatcttgaacggtgaaatttaagaaatggagacaataaatttatgagattaattctgattgaaatgagtaaactaaacacaaatttgaagctttagtttcaattcacatgttttatacaatgggcagggagctagaatataagtccatccgaaaaattgttaaagaggtctataaaaggattagttgcattcctttacacattgctgataatccaattgggctagagcatgcagtgctagaagtgaaatctctccttggacatggttctgatgtcaacattataggaatttatggtattggtggtatcggtaaaaccacaatttctcgtgccgtgtataacttgatttgtagtcaatttgaaggcacatgttttctccttgacattagagaaaaggcaatcaataagcaaggtcttgtccaactccaagaaatgttactttctgaagtactcaagaagaaacatatcaaagtgggtgatgttaacagaggaattccaataataaaaaggaggcttgaaaaaaagaaggttcttctggttttagatgatgttgacaaattagagcagttaaaggttcttgcaggagaaagtcgttggtttggcaatggaagtataatcattatcaccacaagagacaaacacttgctagcgactcatggggtggtaaaatatatgatgttaaaccattaaatgttgcaaaggctcttgaattgtttaactggtgtgcctttaaaaatcataaagctgatccactttatgtgaatattgctaaccgtgcagtttcttatgcgtgtggcattccattggctctggaagtgataggttctcatttatttggaaaaagtttgaatgaatgtaattctgcattggagggcgagccctggtgcagcggtaaagttgtgccttggtgacttgttggtcatgggttcgaatccggaaacagcctctttgcatatgcaagggtaagactgcgtacaatatccctccctcataccttcgcatagcgaagagcctctgggcaatggggtacgaatgtaattctgcattggataaatatgaaagaattcctcatgaaaagatccatgaaatacttaaagtaagctatgatggtttggaagaaaatgagaagcaaattttccttgacattgcttgtttcttcaatacttgtggtgtgggctacgtcacatcagtgctacgtgctcatggttttcatgtaaaagatggtttgagagtgttggttgacagatctctcttaaagattgatgcttctggttgtgtgagaatgcatgatctaattcgagacacaggcagagaaattgtaaggcaagaatcaacagttgagcctggcagacgtagtaggttatggtttgaagaggacattgttcatgttttggaagaaaatacggtatgttagaaattacgaacatctctatgctatttctttgcttttcattcctttattaaaataacaattttactgaatacatgataatttttgggattctttcccctgttatctatttaatttttatatgatgatgagattgctaatgtgcacatttttgtggctaaattgtgctttgggcatggaagggaactgataaaatagaattcataaagcttgaagggtacaacaacatacaagtgcaatggaatggaaaagccttgaaggagatgaagaacctgaggattttgattattgaaaatacaaccttttctacaggccccgagcatctgccaaatagtttgagagtgctagattggagttgctacccttcaccatctttgcctgctgatttcaatcctaaacgagttgagctactccttatgcctgaaagttgccttcaaatcttccaaccatacaatgtatgtatcagtttcgaagttattatttttaacatgaatttctactttaaaaaaaaaaaaaaacacgcacctcacacacaacttgagtcattgtcttttaatttgcagatgtttgaatcattgtctgtgttaagcattgaagattgccagttcttaacagatctaccaagcctacgagaagtaccacttttagcatatttgtgtattgataattgcaccaatttggttaaaattgatggttcaattggctttcttgataagctccaactcttgagtgctaaacggtgtagcaagctaaaaattctagcaccctgcgtcatgttgccatctcttgaaattttggaccttcgggggtgcacatgtcttgatagcttcccggaagtattgggaaagatggagaacataaaagaaatatatttagacgagacagccatagagacactgccatgttcaattggaaattttgttgggcttcaacttttgtccctgaggaaatgcggaaggcttcatcagctaccaggtagtatatgtatattgccgaaagttaaagtgattttcggttttgggcatgtggtatatcgattttgggaagagaaccaatatgaacaagagttgagcttagaagtgtctccaaggtccatggttgttgttgatggtgacctggattttacatatattgacatgtattatccacacataagtccaaataatgtcatccaagtatgcagtcctaatctgctcttgcatcatgatttcattttgttgttcaaaaagcttgcaagcaaggatgggagcagatgcaggaaatcgtcaatgcatttctcgtttcgaaacaaattccccaagattgcggtatgttgttctgtattgcctagtttgaaaattaccatgatattgattttgaagttgggcgtactcatcaacggcactaagatatttagttcatcatgtaactacttatttacacaaacatggaacccaaaactttggtgtgatctagaaagcaaagcagaggggatgttttcagagcaagaatggaatgaagctgagattttgtttgagttgggataccccacaccatgtgctggattaacaatgtcgctccacaactttgctgagggacgtctaaattggaccctatttggtgtctacgaggagggaaataataaggaggatattaaatttaaagatcccatgtcaaccttttcattctgtaacatgctcatggaacctccctcttcattacctagttgtttgtactatgttgttaaatga

SEQ ID NO: 9 - DNA, 20 bp, Artificial Sequence
  GmSINE1-F sequence
gaaagtcgttggtttggcaa

SEQ ID NO: 10 - DNA, 20 bp, Artificial Sequence
  GmSINE1-R sequence
ccatgagcacgtagcactga

(19) United States
(12) Patent Application Publication
(10) Pub. No.: US 2024/0182918 A1
(43) Pub. Date: Jun. 6, 2024

(54) R GENE FOR CONTROLLING MATCHING OF SOYBEAN-RHIZOBIUM, PROTEIN AND USE THEREOF

(71) Applicants: HENAN UNIVERSITY, Kaifeng City, Henan Province (CN); HUAZHONG AGRICULTURAL UNIVERSITY, Wuhan City, Hubei Province (CN)

(72) Inventors: Xuelu WANG, Kaifeng (CN); Bao ZHANG, Kaifeng (CN); Youguo LI, Wuhan (CN)

(21) Appl. No.: 18/004,060

(22) PCT Filed: Dec. 16, 2021
(86) PCT No.: PCT/CN2021/138591

§ 371 Date: Dec. 31, 2022

(30) Foreign Application Priority Data
Dec. 23, 2020 (CN) 202011537197.4

(51) Int. Cl. C12N 15/82; C07K 14/415

(52) U.S. Cl. CPC C12N 15/8261; C07K 14/415

(57) ABSTRACT

A R gene is arranged for controlling soybean-rhizobia compatibility, wherein the GmNNL1 gene of the gene sequence in Hengfeng black bean of the soybean products is shown in SEQ ID NO. 2, and the encoded amino acid sequence is shown in SEQ ID NO. 3. The soybean R gene GmNNL1 is an effective gene that is able to regulate the nodule number of specific rhizobia on soybean, and to regulate the nodule number by directly recognizing the haplotype of the bradyrhizobia-specific effector protein NopP, which is able to limit the symbiotic nodulation of indigenousrhizobia, make soybean preferentially nodule with artificially applied high-efficiency rhizobia agents, and improve symbiotic nitrogen fixation capacity.

### FIELD OF INVENTION

The present invention relates to a technical field of biotechnology for an application of new genes, and more particularly to an R gene for controlling soybean-rhizobia compatibility and its protein thereof and application.

### DESCRIPTION OF RELATED ARTS

Soybean cultivation [Glycine max (L.) Mer] is originated in China and is one of the important foods, feeds, oils and energy crops in the world. It is one of the traditional “grains” and an essential component of people's daily diet (Palander et al., 2005). Meanwhile, with the development of our country economy and the sharp increase of the population's demand for food, our country has used a large number of chemical fertilizers in food production, of which nitrogen fertilizer is the most common chemical fertilizer. According to the statistics of the World Food and Agriculture Organization (FAO), the annual usage of nitrogen fertilizer in our country continuously increases from 2002 to 2014. Although our country's cultivated land area only accounts for 8% of the world, the use of nitrogen fertilizer has reached 35% of the world's total use (http://faostat.fao.org/). Excessive application of nitrogen fertilizers and nutrient loss lead to serious water eutrophication and environmental pollution. Meanwhile, it also affects the composition of soil organic matter, reduces biological activity, and leads to soil compaction. Nitrogen fixation microorganisms can use their own synthetic nitrogenase to reduce nitrogen in the atmosphere to ammonia that can be directly used by plants, that is, biological nitrogen fixation. The symbiotic nitrogen fixation of soybean and rhizobia not only reduces energy consumption (industrial nitrogen fertilizer production consumes more energy) but also improves soil quality, which is the better choice for the of the N supply in agricultural production. In the global ecosystem, the nitrogen fixation by legumes through symbiotic nitrogen fixation accounts for 60%-70% of the biological nitrogen fixation. In the agricultural system, soybean occupies 86% of the symbiotic nitrogen fixation of beans, and the annual nitrogen yield reaches 16.44 million tons (Herridge 2008). Therefore, the symbiotic nitrogen fixation system established by soybean and rhizobia has the great significance to agricultural production and the development of green and sustainable agriculture.

The compatibility is existent between soybean and rhizobia. There are great differences in the nodulation and nitrogen fixation characteristics of the same rhizobia on different soybean lines, or different rhizobia on the same soybean line. The leguminous plant-rhizobia combination with high compatibility and strong symbiotic nitrogen fixation ability can provide excellent effect of symbiotic nitrogen fixation. Rhizobia with broad-host-rang compatibility can be easily utilized in agricultural production. For example, although the bradysoybean rhizobia USDA110 is a well-recognized high-efficiency and broad-spectrum rhizobia, and provide a better nodulate nitrogen fixation in some soybean lines, but it still only has a low symbiotic nitrogen-fixing ability in some soybean lines. The distribution of rhizobia in our country has obvious geographical distribution characteristics. Different types of rhizobia are distributed in different regions of our country. Tian et al. found that the bradyrhizobia accounted for 100% and 99.6% in Northeast China and South China. The bradyrhizobia accounted for 30% and Sinorhizobia 70% in the Huanghuaihai region with alkaline soil. The bradyrhizobia and Sinorhizobia each account for about 50% in the semi-arid alkaline soil area of Xinjiang (Tian et al., 2012). Studies have shown that the inoculation of Rhizobium can significantly improve the symbiotic nitrogen fixation ability of legume crops, promote root growth and increase crop yield (Ferreira et al., 2009). In major soybean-producing countries such as the United States and Brazil, inoculation of the compatible rhizobia to soybean has been widely promoted and applied as one of the important measures to decreasing fertilizer and increase soybean production (Mendes et al., 2003; Sogut 2006; de Freitas et al., 2012). Although the area inoculated with rhizobia in China is relatively small, we still achieved good benefits therefrom (Tang et al. 2011). Hailong City, Heilongjiang in China took the lead in inoculating 80% of rhizobia in soybean cultivating areas, increasing the yield per mu by more than 10%. However, the total area of soybean inoculated with rhizobia in China has decreased in the past ten years. The inoculated area of rhizobia is less than 3% of the soybean cultivating area, which is far from the international level. An important reason is that the nodulation ability of rhizobia agents in the field is weaker than that of indigenous rhizobia. Soybean originated in China, and the soil in the main soybean cultivating area generally has a large number of adaptability and strong nodulation ability. Since the indigenous rhizobia has low nitrogen-fixing ability (Wu, et al 2017; Sun et al., 2004), it affected the nodulation and efficacy of rhizobia agents. Therefore, limiting the nodulation of indigenous rhizobia in soybean is important for the efficacy of the inoculated high-efficiency rhizobia agents. However, research and technical inventions on how to restrict indigenous rhizobia to nodulate soybeans and then let the high-efficiency rhizobia agents preferentially nodulate the soybeans have not been reported.

### SUMMARY OF THE PRESENT INVENTION

The present invention provides an R gene for controlling soybean-rhizobium symbiotic compatibility, and its protein thereof and application to solve the regulation of soybean and rhizobium symbiotic compatibility, and to provide a solution of how to use soybean and rhizobium symbiotic compatibility to improve soybean nitrogen fixation ability, so as to improve the nitrogen utilization rate of soybean.

The technical solution of the present invention is realized as follows: an R gene for controlling soybean-rhizobia compatibility, wherein the gene sequence of R gene is shown in SEQ ID NO.2 or the modified nucleotide sequence of SEQ ID NO.2. Further, the modification includes insertion of a transposon, deletion or addition of bases, or CRISPR-Cas9 modification. The sequence of the R gene can also be shown in SEQ ID NO.4, SEQ ID NO.5, SEQ ID NO.6, SEQ ID NO.7 or SEQ ID NO.8.

For the functional marker primer pair GmSINE1-F/R used to identify the above mentioned R gene, the sequence of GmSINE1-F is shown in SEQ ID NO.9, and the sequence of GmSINE1-R is shown in SEQ ID NO.10. For the functional marker primer pair GmSINE1-F/R, the amplified band is 443 bp when used to amplify the R gene of SEQ ID NO.2, and the amplified band is 622 bp when used to amplify the R gene of whose sequence shown in SEQ ID NO.4, SEQ ID NO.5, SEQ ID NO.6, SEQ ID NO.7 or SEQ ID NO.8.

The protein is encoded by the above mentioned R gene. Further, its amino acid sequence is shown in SEQ ID NO.3. The nucleotide sequence of the promoter of this gene is shown in SEQ ID NO.1.

The application of the above mentioned R gene or the protein encoded by the R gene is arranged for improving the soybean and soybean-rhizobia combination, weakening and limiting the nodulation of indigenous rhizobia on soybean, enhancing the compatibility of soybean and rhizobia inoculants, and enhancing symbiotic nitrogen fixation ability.

The application of the above mentioned R gene and its promoter is arranged for improving the symbiotic nitrogen fixation of legume crops. Further, its steps are: construct the RNAi carrier of the overexpression vector or the R gene that includes the sequence of R gene as shown in SEQ ID NO.2 and the sequence of the promoter as shown in SEQ ID NO.1, through Agrobacterium-mediated transformation, screen the transgenic positive plants for cultivation, inoculate with rhizobia agents, continue to cultivate and complete the application of enhancing the symbiotic nitrogen fixation ability of soybean.

The present invention has the following advantages.

1. The soybean R gene GmNNL1 of the present invention is obtained by the genome-wide association analysis of the symbiotic nitrogen fixation phenotype of cultivated soybean. Using genetic population verification and soybean hair root transient transformation system, stable transgenic plants and HR reaction identification experiments are successfully isolated, cloned and verified. The soybean R gene GmNNL1 is an effective gene that can regulate the nodule number of on soybean inoculated with specific rhizobia. It can regulate the nodule number by directly recognizing the haplotype of the bradyrhizobia-specific effector protein NopP, which can limit the nodulation of indigenous rhizobia, make soybean preferentially nodulate with artificially applied high-efficiency rhizobia agents, and improve symbiotic nitrogen fixation capacity. For any unclear of the fine regulation of soybean-rhizobium symbiotic compatibility by R gene and the molecular mechanism for increasing the above-ground biomass of crops, this gene participates and inhibit root hair infection and regulating nodulation, thereby affecting the biomass of above-ground parts.

2. The soybean with the GmNNL1^{HT1} haplotype of the functional R gene of the present invention can limit the nodulation of the widely distributed bradyrhizobia with the NopP^{USDA110/USDA6}. However, it cannot limit the nodulation of other rhizobia. The GmNNL1^{HT2-HT6} haplotype with a non-functional R gene (encoding an incomplete structure of the TIR-NBS-LRR protein) cannot restrict the nodulation of rhizobia with any NopP genotypes, thereon. The overexpression of GmNNL1 gene driven by the GmNNL1 gene promoter (SEQ ID. NO: 1 sequence) in soybean plants reduce the nodule number of soybean inoculated with specific rhizobia for easy to nodulate with the inoculated high-efficiency soybean rhizobia inoculum, which can improve the symbiotic nitrogen fixation ability of soybean. Alternatively, the functional GmNNL1 gene can be RNAi or knocked out, such that the transgenic soybean can nodulate with the indigenous rhizobia and improve the symbiotic nitrogen fixation ability (although the efficiency is low).

3. The broken of GmNNL1-NopP recognition relationship can increase the nodule number per plant, the nitrogenase activity and the fresh aboveground biomass per plant. This research will provide genetic resources for nitrogen-efficient molecular breeding of legumes and provide molecular modification technology for the utilization and application of legume-rhizobia, so as to provide the development of environment-friendly green and sustainable agriculture.

### BRIEF DESCRIPTION OF THE DRAWINGS

FIG. 1 is a schematic diagram of the protein structure of each soybean GmNNL1 haplotype.

FIG. 2 illustrates a comparative analysis of the nodule number per plant of each soybean GmNNL1 haplotype.

FIG. 3 illustrates a comparative analysis of the nitrogenase activity (a) and the fresh aboveground biomass per plant (b) of the GmNNL1^{HT1} haplotype soybean product with respect to the GmNNL1^{HT2-HT6} haplotype soybean product.

FIG. 4 illustrates the nodule number of several soybean accessions carrying GmNNL1^{HT1} or GmNNL1 with a GmSINE1 insertion inoculated with USDA110 or the USDA110ΔnopP mutant.

FIG. 5 illustrates that GmNNL1^{HT1} can recognize NopP^{USDA110} to induce HR response in tobacco leaves, but GmNNL1^{HT2} cannot.

FIG. 6 is a chart illustrating an analysis of the nodule number (a) and the analysis of the expression (b) of the soybean pGmNNL1::GmNNL1 stable transgenic lines.

FIG. 7 is a chart illustrating a comparative analysis of the nodule number of hairy roots of RNAi-GmNNL1 transgenic soybean and transgenic soybean with empty vector.

FIG. 8 is a chart illustrating an expression level of GmNNL1 gene in the nodules of RNAi-GmNNL1 transgenic soybean hairy roots.

FIG. 9 is a map showing the distribution of bradyrhizobia with different NopP haplotype in our country.

FIG. 10 is a chart illustrating the nodule number of soybeans with different GmNNL1 haplotypes inoculated with bradyrhizobia with different NopP haplotypes.

FIG. 11 shows that GmNNL1^{HT1} can recognize NopP^{USDA110/USDA6} (but not NopP^{USDA122}) to trigger HR response in tobacco leaves.

### DETAILED DESCRIPTION OF THE PREFERRED EMBODIMENT

The present invention will be clearly and completely described below with reference to the accompanying drawings and embodiments. Obviously, the described embodiments are only some, but not all, embodiments of the present invention. Based on the embodiments of the present invention, all other embodiments obtained by those of ordinary skill in the art without creative efforts shall fall within the protection scope of the present invention.

### Embodiment 1

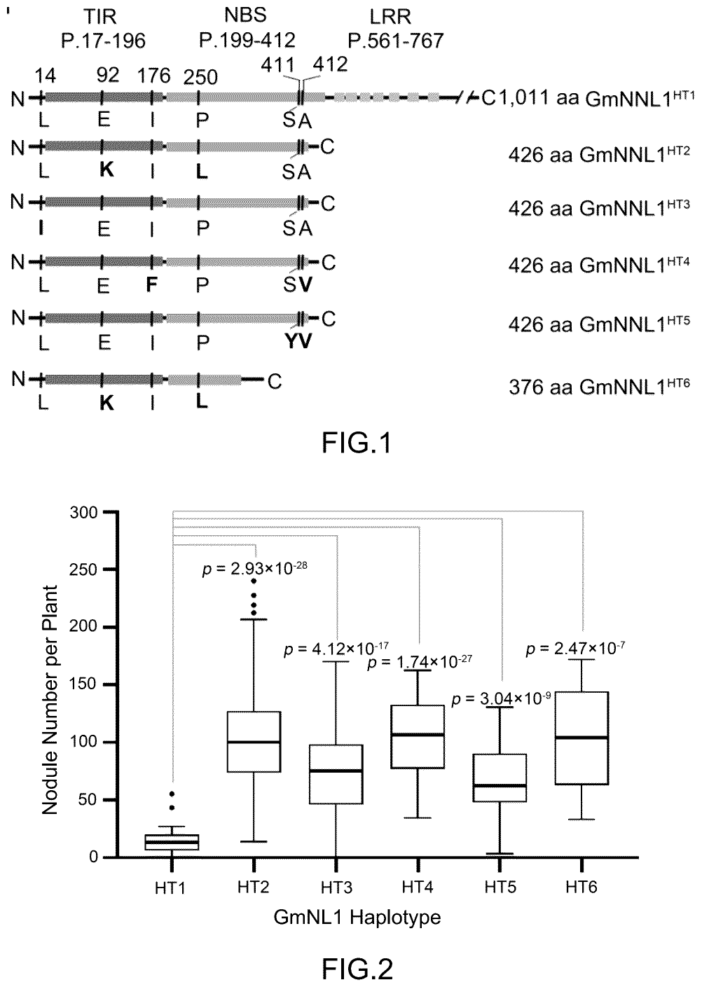

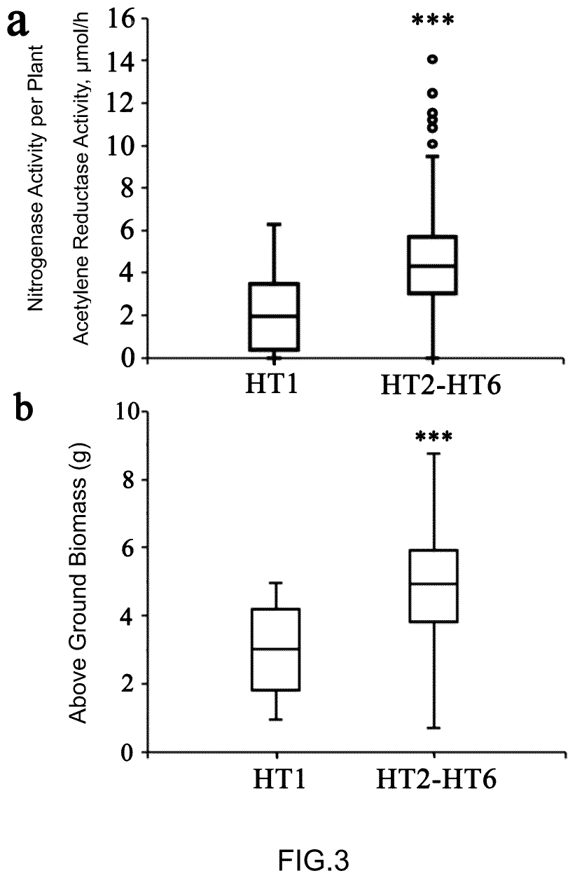

The main objective of the present invention is the core of soybean cultivation. Through genome-wide association analysis, an R gene in soybean, GmNNL1, that can simultaneously affect the number of soybean nodules, nitrogenase activity per plant and above-ground biomass was identified. Through haplotype analysis, it was found that among the 6 main haplotypes of GmNNL1, only GmNNL1^{HT1} (sequence shown in SEQ ID No. 1) could normally encode a complete typical R protein of TIR-NBS-LRR. The other five haplotypes (sequences shown in SEQ ID NO.4, SEQ ID NO.5, SEQ ID NO.6, SEQ ID NO.7 or SEQ ID NO.8) are all caused by the insertion of a transposon GmSINE1 or deletion of a single base resulted in an incomplete protein form (FIG. 1). Through haplotype analysis, it is found that the number of root nodules in soybean with GmNNL1^{HT1} haplotype is significantly lower than that of other haplotypes (FIG. 2). By comparing the complete form of GmNNL1^{HT1} with the incomplete form of GmNNL1^{HT2-HT6} of R protein, it is found that the nitrogenase activity per plant and the fresh weight of the aerial part of soybean with GmNNL1^{HT1} haplotype are significantly lower than those of soybean with GmNNL1^{HT2-HT6} haplotype (FIG. 3).

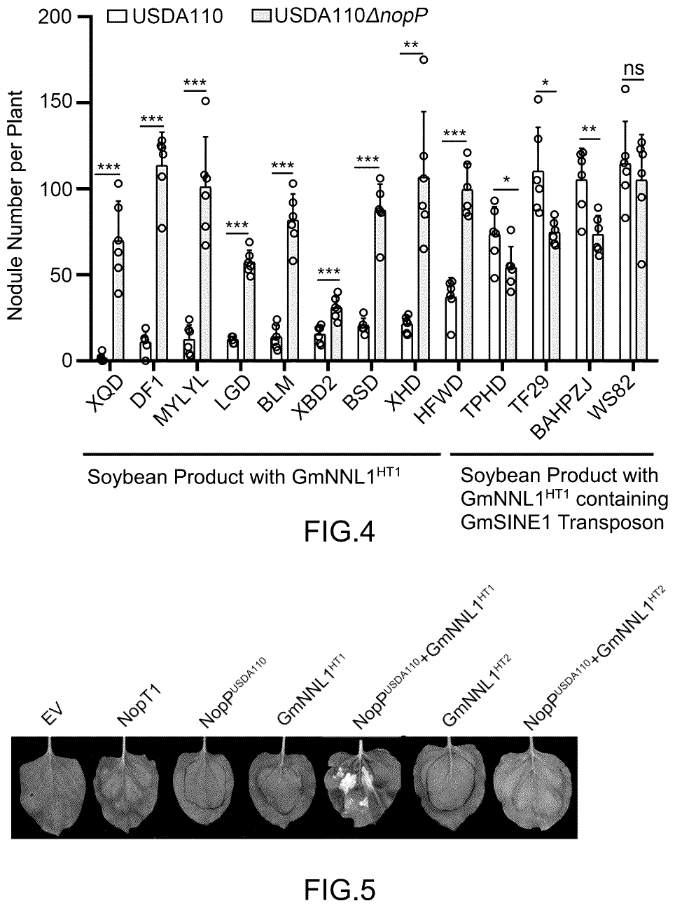

In our study, we found that GmNNL1^{HT1} recognizes the effector protein NopP in Rhizobia. Using USDA110 and its nopP gene mutant strain USDA110ΔnopP, soybean products with GmNNL1^{HT1} and soybean products with GmNNL1 (with GmSINE1 transposon insertion) haplotype are inoculated respectively. It was found that USDA110ΔnopP is able to form more nodules on soybean products with GmNNL1^{HT1} than inoculated with wild-type strain USDA110. However, the nodule number is reduced or not significantly changed on products with GmNNL1^{HT2-HT6} (FIG. 4).

Transient injections of N. benthamiana are injected with NopP^{USDA110}, NopT1 (as a positive control, which has been reported to cause HR-like cell death in tobacco), GmNNL1^{HT1}, GmNNL1^{HT2} alone, NopP^{USDA110} and GmNNL1^{HT1} co-transformed, and NopP^{USDA110} and GmNNL1^{HT2} co-transformed. Co-transfection of NopP^{USDA110} with GmNNL1^{HT1} is found to cause strong HR-like cell death in tobacco leaves after 7 days. However, the transformation of NopP^{USDA110}, GmNNL1^{HT1}, and GmNNL1^{HT2} alone and the co-transformation of NopP^{USDA110} and GmNNL1^{HT2} do not cause strong cell death (FIG. 5), wherein it is indicated that GmNNL1^{HT1} can recognize NopP^{USDA110} and cause HR response.

The specific soybean-rhizobium symbiotic compatibility relationship as: Soybean with the GmNNL1HT1 haplotype of the functional R gene can limit the nodulation of the bradyrhizobia with the NopP^{USDA110} haplotype on it, but it cannot limit the nodulation of the rhizobia on it. The GmNNL1^{HT2-HT6} haplotype with a non-functional R gene (encoding an incomplete structure of the TIR-NBS-LRR protein) is not able to restrict rhizobia nodulation thereon. For the sequences such as 5 haplotypes shown in SEQ ID NO.4, SEQ ID NO.5, SEQ ID NO.6, SEQ ID NO.7 or SEQ ID NO.8, since the typical R protein cannot be encoded into a complete TIR-NBS-LRR, it cannot or is unable to fully exert the function of R protein.

### Embodiment 2

The gene sequence of GmNNL1 is obtained using the following method.

The total volume of the reaction system is 50 μl, and the template is 1 μL (about 50 ng) of genomic DNA of soybean strain Hengfeng black beans, 5 μl of 10×KOD enzyme reaction buffer, 2 μl of 25 mM MgCL2, 5 μl of 5 mM dNTP, 5 μl of 5 μM primers (primers NNL1-F and NNL1-R, each primer is 2.5 μl), 1 μl of KOD enzyme. Add ddH2O (sterile deionized water) to 50 μl. The reaction process is: denaturation at 94° C. for 5 min, 94° C. for 30 sec, 55° C. for 1 min, 68° C. for 3.5 min for 35 cycles, and extension at 68° C. for 10 min. The primers are: NNL1-F: ATGGCACACAGAACAGCACCATCT; NNL1-R: TCATTTAACAACATAGTACAAAC. Finally, a gene sequence containing the nucleotides described in SEQ ID NO.2 is obtained, and the protein encoded by the gene is shown in SEQ ID NO.3.

The protection of the present invention not only includes the nucleotide sequence corresponding to the amino acid sequence shown in SEQ ID NO.3, but also includes the modified protein with the same function of the sequence shown in SEQ ID NO.3.

### Embodiment 3

The application of GmNNL1 gene in regulating soybean-rhizobium symbiotic compatibility and thus affecting the nitrogen fixation efficiency of soybean symbiosis. The application process is as follows:

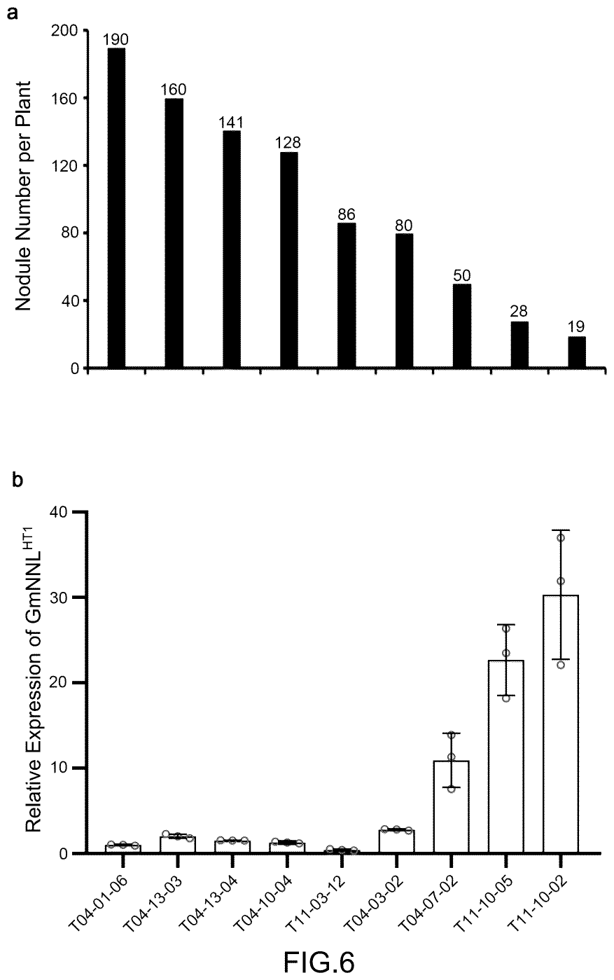

The promoter and full-length CDS of GmNNL1 cloned from HFWD are cloned into pUB-GFP and transferred into Williams82 (WS82, GmNNL1^{HT2}). After inoculation of USDA110 in the obtained transgenic lines, the higher the expression level of GmNNL1^{HT1}, the less the nodule number (FIG. 6). Therefore, the nodulation of specific species of rhizobia can be reduced by this method. The specific process is:
(1) The GmNNL1 promoter region is obtained by the following method.

The total volume of the reaction system is 50 μl, and the template is 1 μl (about 50 ng) of Hengfeng black beans genomic DNA, 5 μl of 1×KOD enzyme reaction buffer, 2 μl of 25 mM MgCL2, 5 μl of 5 mM dNTP, and 5 μl of 5 μM primer (primers pNNL1-F and pNNL1-R are 2.5 μl respectively), 1 μl of KOD enzyme. Add ddH2O (sterile deionized water) to 50 μl. The reaction program is: denaturation at 94° C. for 5 min, 94° C. for 30 sec, 55° C. for 1 min, 68° C. for 2 min for 35 cycles, and extension at 68° C. for 10 min. The primers are as follows: pNNL1-F: TCGTTCCCTCTCATGTGTTCGA; pNNL1-R: GATTTAGGAACTTCAGAAAT. Finally, the promoter region containing GmNNL1 gene shown in SEQ.ID.No.1 is obtained.
(2) Construction of plant expression vector pGmNNL1::GmNNL1

First, the promoter region and gene region of GmNNL1 are amplified from the Hengfeng black beans genome, and the fragment is connected to the pUB-GFP vector to obtain the plant expression vector pUB-pGmNNL1::GmNNL1, then transferred to Williams82 (WS82).

The primers used are as follows:

NNL1-aF:
CAGTGCCAAGCTGGGCTGCAGTCGTTCCCTCTCATGTGTTCGA;
NNL1-aR:
GTCCTTATAGTCCATGGTACCTTTAACAACATAGTACAAACAACT.

(3) Genetic transformation of soybean

According to the present invention, soybean transformation is carried out by Agrobacterium EHA105-mediated transformation of soybean cotyledon nodes, mainly referring to the previously reported transformation method (Luth et al 2015), and improvements are made on this basis.

### 1) Sterilization and Germination of Soybean Seeds

\textcircled{1} Seed sterilization: Pick soybeans without damage and spots and sterilize them with chlorine gas (measure 100 ml of sodium hypochlorite into a 250 ml beaker, slowly add 5 ml of concentrated hydrochloric acid along the wall of the beaker, and seal and sterilize for 16 hours).

\textcircled{2} Seed germination: Place the sterilized soybean seeds in the germination medium, and place them in a 22° C. incubator for 16-24 hours in the dark to germinate.

### 2) Activation of Agrobacterium and Preparation of Infection Solution

Take 250 μl of the frozen bacterial solution and spread it on the LB plate with the corresponding antibiotic, and culture it at 28° C. overnight. The bacterial membrane is scraped by a disposable inoculating loop, suspended in the liquid co-culture medium, and the concentration of the bacterial liquid is measured with a spectrophotometer to make the final concentration OD600=0.5-0.6.

### 3) Explant Preparation and Infection

Retain the hypocotyls with a length of 3-5 mm, separated the two cotyledons, remove the seed coats, and after the primary buds are excised, obtain the cotyledonary node explants for transformation by making several cuts at the cotyledonary node with a blade. Place it in the infection solution and oscillate on a horizontal rotator (rotation speed 50-80 r/min) to infect for 30 min.

### 4) Co-Culture

Pour off the bacterial liquid, transfer the explants to a solid co-culture medium covered with a layer of sterile filter paper, 15-20 per dish, and cultivate in a dark incubator at 22° C. for 3-5 days.

### 5) Screening Culture and Plant Regeneration

\textcircled{1} Bud induction culture: After co-cultivation for 3 to 5 days, the explants are transferred to the bud induction medium, 5 explants are placed in each dish, and the photoperiod is 16/8 hours (light/dark), cultured at 25° C., subculture once every 2 weeks, and subculture twice.

\textcircled{2} Bud elongation culture: remove dead buds, cut off the cotyledons, transfer the explants to bud elongation medium, put 5 explants per dish, photoperiod 16/8 hours (light/dark), culture at 25° C., subculture once every 3 weeks, subculture 2 to 4 times.

\textcircled{3} Rooting induction: When the elongated seedlings grow to 3 cm long, cut and transfer to root induction medium, and cultivate under the conditions of photoperiod 16/8 hours (light/dark) at 25° C.

### 6) Seedling Refining and Transplanting

\textcircled{1} When the regenerated plant grows root and grows two or more compound leaves, take out the plant, wash the medium at the root, and plant it in a small flowerpot filled with sterilized vermiculite. The cycle is 16/8 hours, the relative humidity is 85% RH, and the light intensity is 90 μM/m2/s) for 5 to 7 days.

\textcircled{2} After the seedlings grow stronger, transplant them into large flowerpots (nutrient soil:vermiculite=1:1), and transfer to the culture room (temperature 28±2° C., photoperiod 13.5/10.5 h, relative humidity 40%-60% RH, light intensity 90 μM/m2/s) to grow to until maturity.

### 7) Identification of Plants in T1 Generation

Extract DNA from the leaves of the T1 generation transgenic soybean plants, and amplify all the T1 generation plants by PCR and electrophorese using the functional marker GmNNL1^{HT1} gene GmSINE1-F/R. The single plant that can amplify the GmNNL1^{HT1}-specific 443 bp band is the successful transgenic plant.

The primers are as follows:

GmSINE1-F:
GAAAGTCGTTGGTTTGGCAA;
GmSINE1-R:
CCATGAGCACGTAGCACTGA.

Note: The protection of the present invention also includes the functional marker GmSINE1-F/R of GmNNL1^{HT1}. GmSINE1-F/R as a functional marker of GmNNL1^{HT1}, a fragment of 443 bp can be amplified in the genomic DNA of the line with GmNNL1^{HT1}, and a fragment of 622 bp can be amplified in the genome of soybean products with GmNNL1^{HT2-HT6}. It can be used for the identification and molecular marker-assisted selection of GmNNL1 haplotypes.
(4) Identification and phenotype analysis of GmNNL1HT1 expression in transgenic plants

Twenty-five days after inoculation with rhizobia USDA110, inspect the nodule number of transgenic plants, and analyze the gene expression of GmNNL1^{HT1} in nodules by Real Time qRT-PCR method. The primers are as follows:

GmNNL1^{HT1}_qF1:
GAATGTAATTCTGCATTGGA;
GmNNL1^{HT1}_qR1:
CACCACAAGTATTGAAGAAACA.

### Embodiment 4

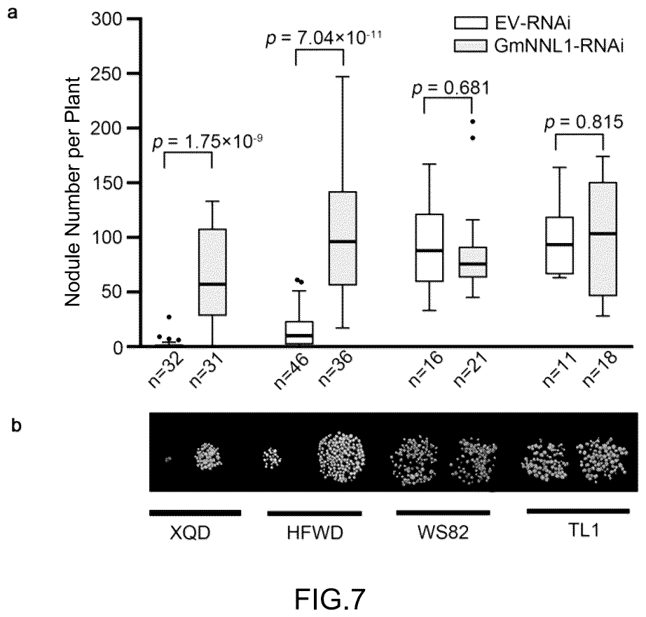

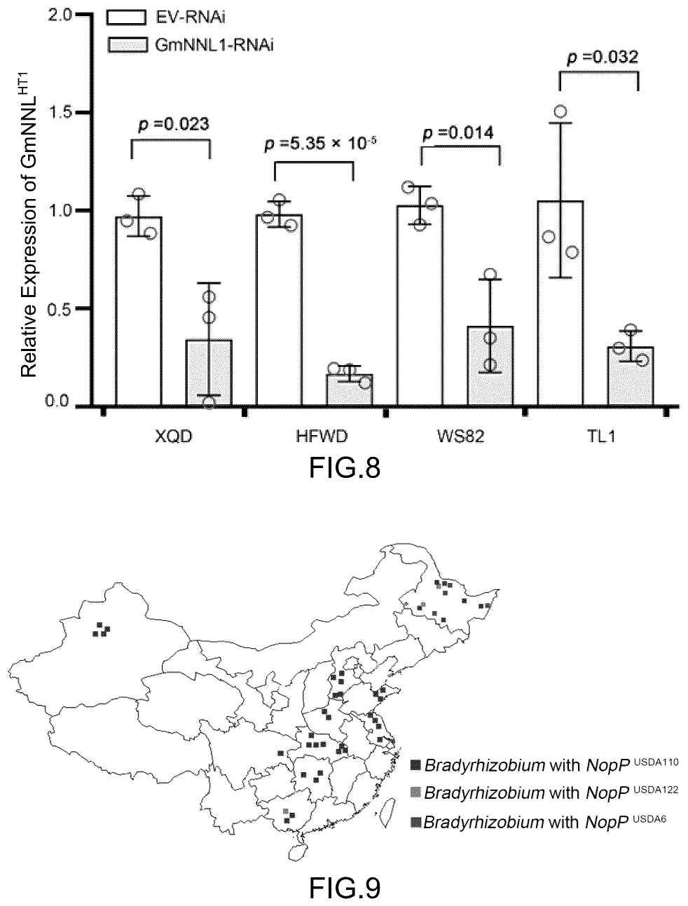

Construction and application of GmNNL1 gene interference vector: proceed the GmNNL1 gene in Hengfeng black bean of the soybean products (HFWD, GmNNL1^{HT1}), small green beans (XQD, GmNNL1^{HT1}), Williams82 (WS82, GmNNL1^{HT2}) and Tianlong No. 1 (TL1, GmNNL1^{HT2}) to RNAi in the hair root transformation system. After the obtained transgenic hair roots are inoculated with USDA110, the expression of GmNNL1^{HT1} decreased, and the nodule number increased in the hair roots of HFWD and XQD. In the hair roots of WS82 and TL1, the nodule number did not change significantly after the expression of GmNNL1^{HT2} is decreased (FIG. 7 and FIG. 8).
(1) Construction of plant expression vector RNAi-GmNNL1: A fragment of 296 bp (+2804-+3099 bp) of GmNNL1 gene is amplified from Hengfeng black beans, and digested using AscI/SwaI and AvrII/BamHI restriction sites, and is connected to RNAi vector pG2RNAi2 to construct pG2RNAi2-GmNNL1 (GmNNL1-RNAi) recombinant vector.

The primers are as follows: 2R-RNAi-F:

2R-RNAi-F:
CGCCTAGGGGCGCGCCAGGAAATGCGGAAGGCTTCA,
2R-RNAi-R:
CGGGATCCATTTAAATCTCTGCTCCCATCCTTGCTT.

(2) Transformation of soybean hair roots mediated by Agrobacterium rhizogenes K599

### 1) Sterilization and Germination of Soybean Seeds

\textcircled{1} Seed sterilization: Pick soybeans without damage and spots and sterilize them with chlorine gas (measure 100 ml of sodium hypochlorite into a 250 ml beaker, slowly add 5 ml of concentrated hydrochloric acid along the wall of the beaker, and seal and sterilize for 16 hours).

\textcircled{2} Seed germination: Sow the sterilized soybean seeds in sterilized quartz sand, place in a soybean artificial climate chamber for 4 to 5 days, and grow until the cotyledons are erect and are at a right angle to the hypocotyl.

2) Preparation of Agrobacterium rhizogenes with the Target Recombinant Vector

Introduce the recombinant plasmid into Agrobacterium rhizogenes K599 to obtain engineered bacteria; activate the engineered bacteria in a liquid medium, take 500 μl of engineered bacteria liquid and then culture at 28° C. for about 2 to 3 days to form a “biofilm”.

### 3) Infection

\textcircled{1} On the 5th day of germination (that is, after 3 days of dark culture and 2 days of light culture), take out the soybean and cut it along 45° at the “green-white junction” of the hypocotyl.

\textcircled{2} Dip the engineering bacteria at soybean hypocotyl incision on the engineering bacteria plate; put the infected soybeans into a nitrogen-free solid medium for co-cultivation for 2 days in the dark.

\textcircled{3} Transfer the plants to square dishes with nitrogen medium, and culture the hypocotyls under the shade for 6 days.

### 4) Hair Root Culture

Remove the plant, and after removing the roots grown at the cut surface callus, place the plants in a nitrogen-containing liquid medium for 9 to 10 days.

### 5) Identification and Screening of Transgenic Positive Roots

\textcircled{1} Take out the plant, absorb the water on its roots by absorbent paper, and test the positive roots under the asana fluorescence microscope, wherein positive roots are defined with GFP green fluorescence. Only the strongest single positive root is left, and the rest are cut off. The plants with a single positive root are continued to be cultured in nitrogen-containing FM medium for 4 to 5 days, and then transferred to soil for culture and inoculation.
(3) Identification and phenotype analysis of GmNNL1 expression in hair roots of transgenic plants.

Twenty-five days after inoculation with rhizobia USDA110, inspect the nodule number in the hair roots of the transgenic plants, and analyze the expression of GmNNL1 in the nodules by Real Time qRT-PCR.

The primers are as follows: GmNNL1^{HT1}_qF1/qR1 for HFWD and XQD, GmNNL1^{HT2}_qF1/qR1 for detection of GmNNL1 gene expression in WS82 and TL1 transgenic hairy roots.

GmNNL1^{HT1}_qF1:
GAATGTAATTCTGCATTGGA;
GmNNL1^{HT1}_qR1:
CACCACAAGTATTGAAGAAACA;
GmNNL1^{HT2}_qF1:
GAGCCCTGGTGCAGCGGTAAAGTTGT;
GmNNL1^{HT2}_qR1:
GGCTCTTCGCTATGCGAAGGTATGAGGGA.

### Embodiment 5

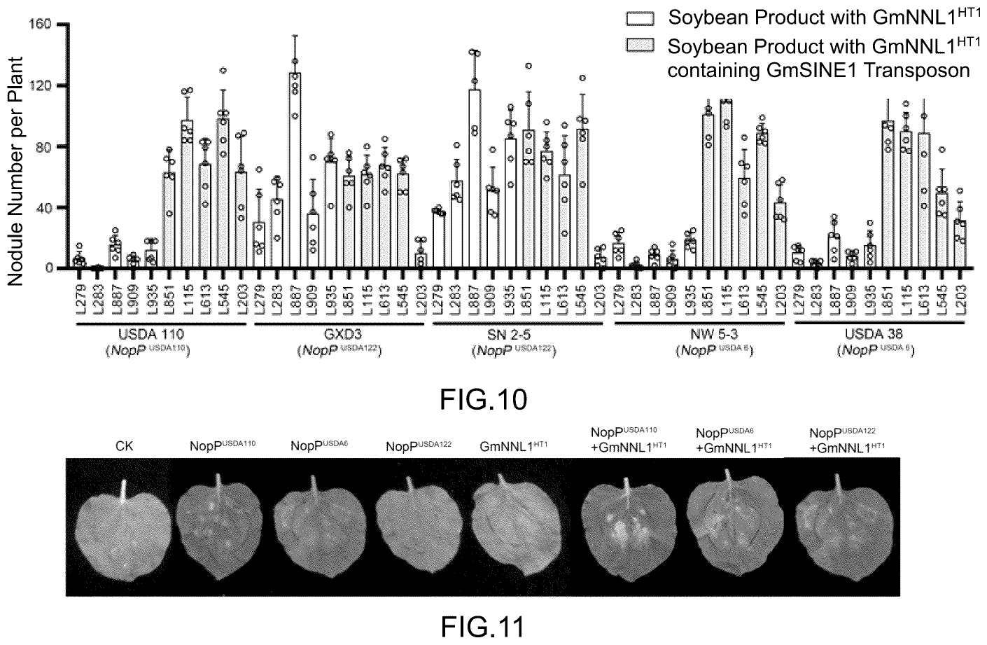

(1) Through the detection of NopP haplotypes of 154 indigenous rhizobia strains in different regions of our country, three NopP haplotypes are mainly identified in our country, wherein 144 strains (93.50% of the total detected rhizobia) have NopP^{USDA110}, 5 strains (3.25% of the total detected rhizobia) have NopP^{USDA6} and 5 strains (3.25% of the total detected rhizobia) have NopP^{USDA122}, and NopP^{USDA110} is found to be the most widely distributed rhizobia type (FIG. 9).
(2) The nodulation performance of rhizobia inoculated with different NopP haplotypes through soybeans with different GmNNL1 haplotypes is shown in FIG. 10. It is found that soybeans with the GmNNL1HT1 haplotype of the functional R gene could restrict the nodulation of indigenous bradyrhizobia bearing the NopPUSDA110/USDA6 haplotype on it. However, it cannot limit the nodulation of other rhizobia on it. The GmNNL1^{HT2-HT6} haplotype with a non-functional R gene (the structure of TIR-NBS-LRR is incomplete) is not able to restrict rhizobia nodulation thereon.

Specifically: the choice of soybean material. Ten soybean materials are selected in the present invention, wherein 5 soybean materials have functional GmNNL1^{HT1}, which are L279, L283, L887, L909 and L935 respectively; the other 5 soybean materials have non-functional forms of GmSINE1 transposon in GmNNL1, L851, L115, L613, L545 and L203.

Selection of rhizobia materials. Five soybean rhizobia strains are selected in the present invention, which are USDA110(NopP^{USDA110}), GXD3(NopP^{USDA122}), SN2-5(NopP^{USDA122}), NW5-3(NopP^{USDA6}) and USDA38(NopP^{USDA6}).
3) NopP^{USDA110}, NopP^{USDA6}, NopP^{USDA122}, GmNNL1^{HT1}, co-transformed GmNNL1^{HT1} and NopP^{USDA110}, co-transformed GmNNL1^{HT1} and NopP^{USDA6}, and co-transformed GmNNL1^{HT1} and NopP^{USDA122} are injected instantaneously in N. benthamiana. After 7 days, it is found that co-transfection of GmNNL1^{HT1} and NopP^{USDA110} and co-transfection of GmNNL1^{HT1} and NopP^{USDA6} could induce strong HR-like cell death in tobacco leaves, while other transfections do not induce strong cell death (FIG. 11). This indicates that GmNNL1^{HT1} can recognize NopP^{USDA110/USDA6} and induce HR response.

The above descriptions are only preferred embodiments of the present invention, and are not intended to be limiting the scope of the present invention. Any modification, equivalent replacement, improvement, etc. made within the spirit and principle of the present invention shall be included within the protection scope of the present invention.

1. An R gene for controlling soybean-rhizobium symbiotic compatibility, wherein its characterized in that: a gene sequence is shown in SEQ ID NO.2 or a modified nucleotide sequence of SEQ ID NO.2.

2. The R gene for controlling soybean-rhizobium symbiotic, as recited in claim 1, wherein the modified nucleotide sequence comprises insertion of a transposon, deletion or addition of bases, or CRISPR-Cas9 modification.

3. The R gene for controlling soybean-rhizobium symbiotic compatibility, as recited in claim 2, wherein the gene sequence is shown in SEQ ID NO.4, SEQ ID NO.5, SEQ ID NO.6, SEQ ID NO.7 or SEQ ID NO.8.

4. The R gene for controlling soybean-rhizobium symbiotic compatibility, as recited in claim 1, further comprising a functional marker primer pair GmSINE1-F/R for identifying the R gene, wherein the GmSINE1-F sequence is as shown in SEQ ID NO.9, GmSINE1-R sequence is shown in SEQ ID NO.10.

5. The R gene for controlling soybean-rhizobium symbiotic compatibility, as recited in claim 1, wherein the R gene is encoded to be protein.

6. The R gene for controlling soybean-rhizobium symbiotic compatibility, as recited in claim 5, wherein the amino acid sequence of the protein is shown in SEQ ID NO.3.

7. The R gene for controlling soybean-rhizobium symbiotic compatibility, as recited in claim 1, further comprising a promoter, wherein a nucleotide sequence of the promoter is shown in SEQ ID NO.1.

8. The R gene for controlling soybean-rhizobium symbiotic compatibility, as recited in claim 1, the encoded protein is arranged for improving soybean and soybean-rhizobia combination, weakening and limiting a nodulation of indigenous rhizobia on soybean, and enhancing soybean and rhizobia inoculants compatibility, and enhancing symbiotic nitrogen fixation capacity.

9. The R gene for controlling soybean-rhizobium symbiotic compatibility, as recited in claim 1, wherein the R gene and its promoter is applied for improving the symbiotic nitrogen fixation of legume crops.

10. The R gene for controlling soybean-rhizobium symbiotic compatibility, as recited in claim 9, wherein the application comprises the step of: constructing an overexpression vector or an RNAi vector of the R gene containing the R gene with the sequence shown in SEQ ID NO.2 and the promoter with the sequence shown in SEQ ID NO.1, through agrobacterium-mediated transformation, screening the transgenic positive plants for cultivation, inoculating with rhizobia inoculants, and continuously cultivating to complete the application of enhancing the symbiotic nitrogen fixation ability of soybean.

11. The R gene for controlling soybean-rhizobium symbiotic compatibility, as recited in claim 2, further comprising a functional marker primer pair GmSINE1-F/R for identifying the R gene, wherein the GmSINE1-F sequence is as shown in SEQ ID NO.9, GmSINE1-R sequence is shown in SEQ ID NO.10.

12. The R gene for controlling soybean-rhizobium symbiotic compatibility, as recited in claim 2, wherein the R gene is encoded to be protein, wherein the amino acid sequence of the protein is shown in SEQ ID NO.3.

13. The R gene for controlling soybean-rhizobium symbiotic compatibility, as recited in claim 2, wherein the R gene and its promoter is applied for improving the symbiotic nitrogen fixation of legume crops, wherein the application comprises the step of: constructing an overexpression vector or an RNAi vector of the R gene containing the R gene with the sequence shown in SEQ ID NO.2 and the promoter with the sequence shown in SEQ ID NO.1, through agrobacterium-mediated transformation, screening the transgenic positive plants for cultivation, inoculating with rhizobia inoculants, and continuously cultivating to complete the application of enhancing the symbiotic nitrogen fixation ability of soybean.

14. The R gene for controlling soybean-rhizobium symbiotic compatibility, as recited in claim 3, further comprising a functional marker primer pair GmSINE1-F/R for identifying the R gene, wherein the GmSINE1-F sequence is as shown in SEQ ID NO.9, GmSINE1-R sequence is shown in SEQ ID NO.10.

15. The R gene for controlling soybean-rhizobium symbiotic compatibility, as recited in claim 3, wherein the R gene is encoded to be protein.

16. The R gene for controlling soybean-rhizobium symbiotic compatibility, as recited in claim 15, wherein the amino acid sequence of the protein is shown in SEQ ID NO.3.

17. The R gene for controlling soybean-rhizobium symbiotic compatibility, as recited in claim 3, further comprising a promoter, wherein a nucleotide sequence of the promoter is shown in SEQ ID NO.1.

18. The R gene for controlling soybean-rhizobium symbiotic compatibility, as recited in claim 3, the encoded protein is arranged for improving soybean and soybean-rhizobia combination, weakening and limiting a nodulation of indigenous rhizobia on soybean, and enhancing soybean and rhizobia inoculants compatibility, and enhancing symbiotic nitrogen fixation capacity.

19. The R gene for controlling soybean-rhizobium symbiotic compatibility, as recited in claim 3, wherein the R gene and its promoter is applied for improving the symbiotic nitrogen fixation of legume crops.

20. The R gene for controlling soybean-rhizobium symbiotic compatibility, as recited in claim 19, wherein the application comprises the step of: constructing an overexpression vector or an RNAi vector of the R gene containing the R gene with the sequence shown in SEQ ID NO.2 and the promoter with the sequence shown in SEQ ID NO.1, through agrobacterium-mediated transformation, screening the transgenic positive plants for cultivation, inoculating with rhizobia inoculants, and continuously cultivating to complete the application of enhancing the symbiotic nitrogen fixation ability of soybean.
